(19) United States
(12) Patent Application Publication
(10) Pub. No.: US 2026/0053414 A1
(43) Pub. Date: Feb. 26, 2026

(54) CARDIAC ELECTRICAL ACTIVITY MONITORING USING A WEARABLE DEVICE

(71) Applicant: Everbeat, Inc., Rockville, MD (US)

(72) Inventor: George Stuart MENDENHALL, Del Mar, CA (US)

(73) Assignee: Everbeat, Inc., Rockville, MD (US)

(21) Appl. No.: 19/304,438

(22) Filed: Aug. 19, 2025

Related U.S. Application Data
(60) Provisional application No. 63/685,150, filed on Aug. 20, 2024.

(51) Int. Cl. A61B 5/282; A61B 5/00; A61B 5/256; A61B 5/308; A61B 5/327; A61B 90/00

(52) U.S. Cl. CPC A61B 5/282; A61B 5/256; A61B 5/308; A61B 5/327; A61B 5/6826; A61B 5/6843; A61B 5/7203; A61B 90/08; A61B 2090/0807; A61B 2560/0468

(57) ABSTRACT

A wearable electronic device for cardiac electrical activity monitoring includes a housing configured to be worn around a first body part of a user, the housing having an inward-facing surface that faces the first body part and an outward-facing surface that faces away from the first body part. The device includes a plurality of electrodes, including at least one inner electrode at the inward-facing surface of the housing for contacting the first body part. The plurality of electrodes also includes multiple outer electrodes at the outward-facing surface of the housing for contacting one or more other parts of a body of the user. The device includes a controller configured to generate a cardiac electrical activity signal using a pair of electrodes selected from the plurality of electrodes based on at least one indicia of contact between an electrode of the pair of electrodes and the user.

### CROSS REFERENCE TO RELATED APPLICATIONS

This application claims the benefit of U.S. Provisional Application No. 63/685,150, filed Aug. 20, 2024, the entire contents of which is incorporated herein by reference.

### FIELD

The present disclosure relates generally to devices and systems for cardiac monitoring, and more specifically to wearable devices for generating data related to electrocardiography.

### BACKGROUND

Diagnostic measurement of electrical activity of the heart is often limited to healthcare facilities such as hospitals and doctors'offices, reducing early detection of life-threatening cardiovascular diseases, given the complexities associated with known electrocardiographic techniques. Such complexities can include placement of electrodes at precise locations and in a manner that ensures proper contact with the patient's skin, in addition to interpretation of resulting data. Wearable devices have been developed that strive to improve access to cardiac monitoring by enabling monitoring of electrical activity of the heart outside of healthcare facilities. Such wearable devices are typically configured with fewer electrodes than conventionally used in a healthcare facility setting to reduce device complexity and increase ease of use. Such wearable devices often require the user to place the device in a particular position and/or orientation on the body to provide accurate measurements. This requirement often leads to user error and poor measurement accuracy.

### SUMMARY

Described herein are improved systems, devices, and methods for cardiac monitoring using wearable devices that include multiple electrodes, automatically detect contact with a user's body at any one of multiple locations corresponding to the electrodes, and select electrodes to use for cardiac electrical activity monitoring based on which electrodes likely provide the best cardiac signal detection. Thus, the wearable monitoring device may be positioned and/or oriented in multiple different ways, increasing the usability and decreasing the likelihood of poor cardiac signal monitoring.

The device may detect contact between the user and different regions of the wearable device corresponding to the electrodes and may select at least one pair of electrodes for measuring electrical activity of the heart based on whether the user is contacting the wearable device in the regions of the electrodes. For example, a particular one of a plurality of electrodes on an outward-facing surface of the wearable device may be selected by a controller of the wearable device for monitoring heart activity based on indicia that the user is contacting the particular electrode. Indicia of contact of an electrode by a user may include, for example, feedback signals from contact sensors such as capacitive touch sensors co-located with one or more electrodes, detection of cardiac electrical activity by a pair of electrodes, and/or detection of noise by a pair of electrodes (thereby providing a negative indication of contact). The controller may determine that an indicia of contact associated with a particular electrode exceeds a contact threshold and may select that particular electrode for heart activity monitoring. The device may compare indicia of contact associated with various electrodes and may select one or more electrodes for heart electrical activity monitoring based on which indicia of contact is higher. By automatically selecting electrodes based on an evaluation of contact with the user, the device improves useability, such as by increasing the likelihood usable data may be obtained irrespective of the orientation of the device.

In some embodiments, a wearable electronic device for cardiac electrical activity monitoring is provided, the device comprising a housing configured to be worn around a first body part of a user, the housing having an inward-facing surface that faces the first body part and an outward-facing surface that faces away from the first body part; a plurality of electrodes comprising at least one inner electrode at the inward-facing surface of the housing for contacting the first body part, and multiple outer electrodes at the outward-facing surface of the housing for contacting one or more other parts of a body of the user; and a controller configured to generate a cardiac electrical activity signal using a pair of electrodes selected from the plurality of electrodes based on at least one indicia of contact between an electrode of the pair of electrodes and the user.

In some embodiments, the controller is configured to generate multiple cardiac electrical activity signals using multiple pairs of the electrodes. In some embodiments, the multiple cardiac electrical activity signals comprise a first cardiac electrical activity signal that is based on an electric potential between a first pair of electrodes, a second cardiac electrical activity signal that is based on an electric potential between a second pair of electrodes, and a third cardiac electrical activity signal that is based on the first cardiac electrical activity signal and the second cardiac electrical activity signal; wherein the first pair of electrodes comprises an inner electrode that contacts the first body part and a first outer electrode that contacts a second body part, and wherein the second pair of electrodes comprises the inner electrode and a second outer electrode that contacts a third body part. In some embodiments, the controller is configured to generate data to simulate an electrocardiogram with at least three leads based on at least one of the first cardiac electrical activity signal, the second cardiac electrical activity signal, or the third cardiac electrical activity signal. In some embodiments, the controller is configured to select the pair of electrodes from the plurality of electrodes by determining that the at least one indicia of contact meets a threshold value. In some embodiments, the controller is configured to select the pair of electrodes from the plurality of electrodes by determining that a first indicia of contact associated with a first electrode of the plurality of electrodes exceeds a second indicia of contact associated with a second electrode of the plurality of electrodes. In some embodiments, the first electrode and the second electrode are both located at the inward-facing surface or both located at the outward-facing surface of the housing. In some embodiments, the wearable electronic device further comprises at least one contact sensor configured to detect contact with the user, wherein the at least one indicia of contact comprises a feedback signal from the at least one contact sensor. In some embodiments, the at least one contact sensor comprises multiple contact sensors, and wherein each contact sensor of the multiple contact sensors is proximate to a respective electrode of the plurality of electrodes. In some embodiments, the wearable electronic device further comprises at least one analog-to-digital converter and at least one switch for selectively connecting different contact sensors to the at least one analog-to-digital converter. In some embodiments, the at least one contact sensor comprises at least one capacitive touch sensor. In some embodiments, the at least one indicia of contact comprises cardiac electrical activity detected via the pair of electrodes and/or a signal to noise ratio corresponding to the detection of cardiac electrical activity by the pair of electrodes. In some embodiments, the pair of electrodes is a first pair of electrodes and wherein the at least one indicia of contact comprises a signal corresponding to noise detected by a second pair of electrodes. In some embodiments, generating the cardiac electrical activity signal comprises recording multiple signals using multiple pairs of electrodes of the plurality of electrodes, before selecting the pair of electrodes, and recorded signal corresponding to the pair of electrodes, from among the multiple pairs of electrodes based on the at least one indicia of contact. In some embodiments, the wearable electronic device further comprises at least one signal-generating circuit and at least one switch for selectively connecting different electrodes to the at least one signal-generating circuit. In some embodiments, the pair of electrodes is a first pair of electrodes, and wherein generating the cardiac electrical activity signal comprises enhancing a signal generated by the first pair of electrodes based on a signal generated by a second pair of electrodes of the plurality of electrodes. In some embodiments, the signal generated by the second pair of electrodes corresponds to noise, and wherein enhancing the signal generated by the first pair of electrodes comprises subtracting the signal generated by the second pair of electrodes. In some embodiments, the controller is configured to generate data suitable for simulating an electrocardiogram with at least one lead based on the cardiac electrical activity signal. In some embodiments, the pair of electrodes comprises an inner electrode opposite to an outer electrode, wherein the inner electrode and/or the outer electrode was selected based on the at least one indicia of contact. In some embodiments, the outward-facing surface of the ring comprises an indication of a position of at least one of the multiple outer electrodes. In some embodiments, the outward-facing surface of the ring does not comprise an indication of positions of any of the multiple outer electrodes. In some embodiments, the device is configured as a ring and the first body part is a finger.

In some embodiments, a method for cardiac electrical activity monitoring using a wearable electronic device is provided, the method comprising selecting a pair of electrodes from a plurality of electrodes of the device based on at least one indicia of contact between an electrode of the pair of electrodes and a user; and generating a cardiac electrical activity signal using the pair of electrodes.

In some embodiments, the method further comprises generating multiple cardiac electrical activity signals using multiple pairs of electrodes. In some embodiments, generating multiple cardiac electrical activity signals comprises generating a first cardiac electrical activity signal based on an electric potential between a first pair of electrodes; generating a second cardiac electrical activity signal based on an electric potential between a second pair of electrodes; and generating a third cardiac electrical activity signal based on the first cardiac electrical activity signal and the second cardiac electrical activity signal; wherein the first pair of electrodes comprises an inner electrode that contacts the first body part and a first outer electrode that contacts a second body part, and wherein the second pair of electrodes comprises the inner electrode and a second outer electrode that contacts a third body part. In some embodiments, the method further comprises generating data to simulate an electrocardiogram with at least three leads based on at least one of: the first cardiac electrical activity signal, the second cardiac electrical activity signal, or the third cardiac electrical activity signal. In some embodiments, selecting the pair of electrodes from the plurality of electrodes comprises determining that the at least one indicia of contact meets a threshold value. In some embodiments, selecting the pair of electrodes from the plurality of electrodes comprises determining that a first indicia of contact associated with a first electrode of the plurality of electrodes exceeds a second indicia of contact associated with a second electrode of the plurality of electrodes. In some embodiments, selecting the pair of electrodes from the plurality of electrodes comprises detecting contact with the user using at least one contact sensor, wherein the at least one indicia of contact comprises a feedback signal from the at least one contact sensor. In some embodiments, selecting the pair of electrodes from the plurality of electrodes comprises detecting cardiac electrical activity using the pair of electrodes and/or detecting a signal to noise ratio corresponding to the detection of cardiac electrical activity using the pair of electrodes. In some embodiments, the pair of electrodes is a first pair of electrodes and wherein selecting the pair of electrodes from the plurality of electrodes comprises detecting a signal corresponding to noise using a second pair of electrodes. In some embodiments, the pair of electrodes is a first pair of electrodes, and wherein generating the cardiac electrical activity signal comprises enhancing a signal generated by the first pair of electrodes based on a signal generated by a second pair of electrodes of the plurality of electrodes. In some embodiments, the signal generated by the second pair of electrodes corresponds to noise, and wherein enhancing the signal generated by the first pair of electrodes comprises subtracting the signal generated by the second pair of electrodes. In some embodiments, the method further comprises generating data to simulate an electrocardiogram with at least one lead based on the cardiac electrical activity signal.

In some embodiments, a method for cardiac electrical activity monitoring using a wearable electronic device is provided, the method comprising recording multiple signals using multiple pairs of electrodes of the device; and selecting at least one signal as a cardiac electrical activity signal from the multiple signals based on at least one indicia of contact between a user and an electrode of a pair of electrodes used to measure the signal.

In some embodiments, selecting the at least one signal from the multiple signals comprises determining that the at least one indicia of contact meets a threshold value. In some embodiments, the at least one signal comprises a first signal, and wherein selecting the first signal from the multiple signals comprises determining that a first indicia of contact associated with the pair of electrodes used to measure the first signal exceeds a second indicia of contact associated with a pair of electrodes used to measure the second signal. In some embodiments, the method further comprises recording at least one contact sensor feedback signal from one or more contact sensors, wherein the at least one indicia of contact comprises the at least one recorded contact sensor feedback signal. In some embodiments, selecting the at least one signal from the multiple signals comprises detecting cardiac electrical activity and/or detecting a signal to noise ratio corresponding to the detection of cardiac electrical activity. In some embodiments, the pair of electrodes is a first pair of electrodes and wherein selecting the at least one signal from the multiple signals comprises detecting a signal corresponding to noise using a second pair of electrodes. In some embodiments, the pair of electrodes is a first pair of electrodes and wherein the method further comprises enhancing a signal generated by the first pair of electrodes based on a signal generated by a second pair of electrodes of the plurality of electrodes. In some embodiments, the signal generated by the second pair of electrodes corresponds to noise, and wherein enhancing the signal generated by the first pair of electrodes comprises subtracting the signal generated by the second pair of electrodes. In some embodiments, the method further comprises generating data to simulate an electrocardiogram with at least one lead based on the cardiac electrical activity signal. In some embodiments, the method further comprises generating a first cardiac electrical activity signal based on an electric potential between a first pair of electrodes; generating a second cardiac electrical activity signal based on an electric potential between a second pair of electrodes; and generating a third cardiac electrical activity signal based on the first cardiac electrical activity signal and the second cardiac electrical activity signal; wherein the first pair of electrodes comprises an inner electrode that contacts the first body part and a first outer electrode that contacts a second body part, and wherein the second pair of electrodes comprises the inner electrode and a second outer electrode that contacts a third body part. In some embodiments, the method further comprises generating data to simulate an electrocardiogram with at least three leads based on at least one of: the first cardiac electrical activity signal, the second cardiac electrical activity signal, and the third cardiac electrical activity signal. In some embodiments, a non-transitory computer readable storage medium storing instructions for cardiac electrical activity monitoring is provided, wherein the instructions, when executed by one or more processors of an electronic device, cause the device to select a pair of electrodes from a plurality of electrodes of the device based on at least one indicia of contact between an electrode of the pair of electrodes and a user; and generate a cardiac electrical activity signal using the pair of electrodes.

In some embodiments, any of the features of any of the embodiments described above and/or described elsewhere herein may be combined, in whole or in part, with one another. Additional advantages will be readily apparent to those skilled in the art from the following figures and detailed description. The aspects and descriptions herein are to be regarded as illustrative in nature and not restrictive.

### BRIEF DESCRIPTION OF THE FIGURES

A better understanding of the features and advantages of the present disclosure will be obtained by reference to the following detailed description that sets forth illustrative embodiments, in which the principles of the disclosure are utilized, and the accompanying figures of which:

FIG. 1 depicts an exemplary wearable monitoring device including inner and outer electrodes and sensors, according to some embodiments.

FIG. 2 depicts contact between an exemplary wearable monitoring device and multiple parts of a user's body, according to some embodiments.

FIG. 3A depicts a first exemplary circuit diagram of a wearable monitoring device, according to some embodiments.

FIG. 3B depicts a second exemplary circuit diagram of a wearable monitoring device, according to some embodiments.

FIG. 4A depicts a first exemplary method for generating data related to electrocardiography using a wearable device, according to some embodiments.

FIG. 4B depicts a second exemplary method for generating data related to electrocardiography using a wearable device, according to some embodiments.

FIG. 5A depicts a first exemplary configuration of a wearable monitoring device, according to some embodiments.

FIG. 5B depicts a second exemplary configuration of a wearable monitoring device, according to some embodiments.

FIG. 6 depicts an exemplary computing system, according to some embodiments.

### DETAILED DESCRIPTION

Accordingly, disclosed herein are systems, devices, and methods that enable a user to measure their cardiac activity using a wearable device without requiring specialized knowledge or technical expertise. An exemplary device may take the form of a ring or bracelet with inner and outer electrodes that detect electric potentials at individual electrodes or pairs of electrodes. The device may automatically detect contact with the user's body, basing the detection on one or more indicia of contact. Indicia of contact may include, for example, detection of a user's contact by a capacitive touch sensor associated with an electrode, detection of cardiac electrical activity above a certain signal to noise threshold, or detection of a signal corresponding to noise at one or more other electrodes. The device may optionally enhance electric potentials by removing detected common mode noise. Based on the indicia of contact, the device may then select a pair of electrodes for use in generating a cardiac signal. The device may selected multiple different pairs of electrodes that contact different parts of the body to generate multiple cardiac signals and may use the generated cardiac signal(s) to simulate an electrocardiogram. Thus, by detecting user contact and selecting electrode pairs without user intervention or input, the disclosed systems, devices, and methods enable a layperson to monitor their cardiac electrical activity, potentially enabling early detection of cardiovascular anomalies.

Wearable devices described herein may have several advantages over conventional cardiac monitoring devices. For example, non-wearable electrocardiography devices require advanced knowledge of electrode placement locations and technique, which may limit ability for laypeople to conduct measurements on themselves. Conventional wearable devices that monitor the electrical activity of the heart require specific positioning and/or orienting of the device, making them more difficult to use and more prone to generating poor cardiac signals.

In the following description of the various embodiments, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed terms. It is further to be understood that the terms “includes,” “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include method steps and instructions described herein in the form of an algorithm. It should be noted that the method steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, storage medium, such as, but not limited to, any type of disk, including floppy disks, USB flash drives, external hard drives, optical disks, CD-ROMs, magneto-optical disks, read-only memories (ROMs), random access memories (RAMs), EPROMs, EEPROMs, magnetic or optical cards, application-specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The structure for a variety of these systems will appear in the description below. In addition, the present invention is not described with reference to any particular programming language. It will be appreciated that a variety of programming languages may be used to implement the teachings of the present disclosure as described herein.

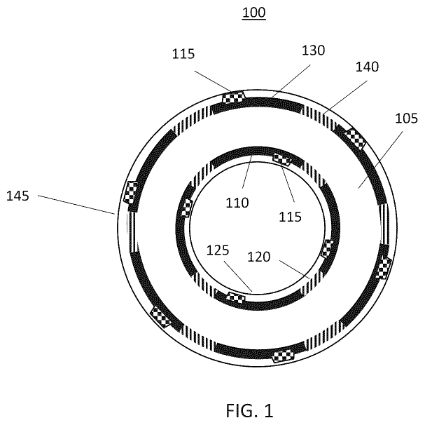

FIG. 1 depicts an exemplary wearable device 100 that can be used to monitor heart electrical activity to generate data for electrocardiography. Wearable device 100 can be annular in shape and can take the form of a ring for wearing on a finger or a bracelet for wearing on a wrist or ankle. Device 100 may include housing 105 configured to be worn around a body part of a user. Housing 105 may include an inward-facing surface 125 facing the body part of the user on which wearable device 100 is worn and outward-facing surface 145 facing away from the body part of the user on which wearable device 100 is worn. Device 100 includes a plurality of electrodes and is configured to measure heart electrical activity using pairs of the electrodes. The electrodes include one or more inner electrodes 110 at inward-facing surface 125 for contacting the portion of the user's body on which device 100 is worn. The electrodes include a plurality of outer electrodes 130 at outward-facing surface 145 that can be contacted by one or more other portion of the user's body. Electrical insulators 140 and 120 may be placed between and/or around electrodes on the outward-facing and inward-facing surfaces respectively to electrically isolate each electrode. Gaps may be used in addition to or in place of electrical insulators thereby providing or improving the electrical isolation of the electrodes. Cardiac electrical activity can be measured, for example, based on the difference in electrical potential between the body part on which device 100 is worn and another body part touching outward-facing surface 145 of device 100 via one of the inner electrodes 110 and one of the outer electrodes 130 or between two different body parts touching outward-facing surface 145 of device 100. As discussed in detail below, device 100 may be configured to select a pair of electrodes to detect cardiac electrical activity based on a determination that the electrodes of the pair are in contact with the user's body or have the best contact with the user's body.

Each electrode may be configured to capture cardiac electrical activity, for example electric potentials necessary to visualize one or more phases of cardiac action potential and associated flow of ions. Electrodes may be composed of a conductive material, such as silver, silver chloride, and/or stainless steel. Electrical insulators 120 and 140 may be composed of an insulative material such as a resin and/or a ceramic material. For example, inward-facing and outward-facing surfaces 125 and 145 may be composed of an electrically insulative material into which each electrode is embedded, thereby electrically isolating each electrode. Additionally, or alternatively, housing 105 may be composed of a semi-conductive or conductive material to which electrodes embedded into electrical insulators 120 and 140 are attached, which may ensure each electrode is electrically isolated from all others. Provision of one or more inner electrodes and a plurality of outer electrodes electrically insulated from one another may enable a flexible mode of operation in which, regardless of the orientation of device 100 relative to the user's body, contact may be created with the user's body. Further, device 100 may automatically determine which pairs of electrodes to utilize to generate cardiac electrical activity signals that can be used to simulate an electrocardiogram as discussed below.

In the one or more embodiments, an increased number of electrodes may yield several benefits. For instance, use of a multitude of electrodes, each occupying a relatively small portion of the outward-facing surface, may decrease the susceptibility of each electrode to unintentional contact. For example, in one or more embodiments in which device 100 includes a ring worn on a user's finger, two outer electrodes would each occupy a significant portion of the outward-facing surface. With this increased electrode area would come an increase in the risk of unintentional contact by a finger adjacent to the finger on which the ring is worn, as compared to a ring with four outer electrodes each occupying less of the outward-facing surface. This decrease in susceptibility to unintentional contact with a higher number of electrodes may in turn translate to fewer disruptions to the detection of and/or less noise added to electric potential data. Device 100 may include at least one, at least two, at least three, or at least four inner electrodes. Device 100 may additionally include at least two, at least three, at least four, at least five, at least six, at least seven, or at least eight outer electrodes.

Device 100 may include at least one contact sensor 115 for detecting contact between body parts of a user and regions of the device 100. A feedback signal from contact sensor 115 may form one indicia of contact used by device 100 to determine contact has been made between the user and one or more electrodes proximate to the sensor. Contact sensor 115 may be positioned proximate to one or more inner electrodes 110 and/or outer electrodes 130. One or more contact sensors 115 may include a capacitive touch sensor. Upon detection of a change in capacitance that optionally exceeds a capacitance threshold, the sensor may determine that contact with a user's body part has been made. Contact sensor 115 may include an inductive touch sensor, detecting changes in circuit inductance such that the sensor may determine that contact with a user's body part has been made upon a detection of a change in inductance that optionally exceeds an inductance threshold. Contact sensor 115 may include a capacitive touch sensor, an inductive touch sensor, and/or a sensor detecting changes in capacitance and/or inductance to determine contact with a user. Additional or alternative types of sensors may be used for one or more contact sensor 115 to detect contact between device 100 and the user, including an optical proximity sensor or an ultrasonic proximity sensor. Each sensor may operate similar to a capacitive and/or inductive touch sensor, wherein once a parameter corresponding to contact exceeds a threshold, the sensor may determine contact with a user's body has been made. This threshold may be set to encourage a user to apply sufficient force to the electrode associated with the contact sensor, as insufficient force may limit the electrical contact between the electrode and the user's body part, thereby reducing signal quality for example by reducing the signal to noise ratio.

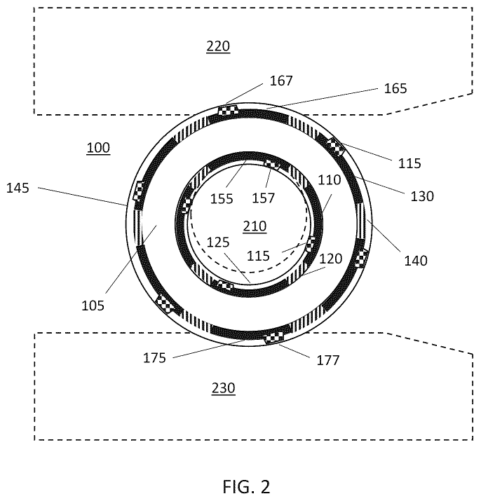

FIG. 2 depicts an exemplary use of wearable device 100. Device 100 is depicted as worn on a body part 210. Device 100 may be in the form of a ring and body part 210 may be a finger. Device 100 may be in the form of a bracelet, anklet, or other ring-shape and body part 210 may be an arm or a leg, such as a wrist or ankle. In FIG. 2, body part 210 is contacting the uppermost inner electrode 155, optionally detected by associated contact sensor 157.

To detect electric potentials at a number of different points and thereby generate data to simulate an electrocardiogram, the user may contact the plurality of outer electrodes 130 of device 100 with any number of parts of their body represented by upper body part 220 and lower body part 230. These parts of the user's body may include the user's leg, arm, wrist, finger, and/or chest. For example, upper body part 220 may include one of the user's arms and lower body part 230 may include one the user's legs, respectively contacting the uppermost and lowermost outer electrodes 165 and 175, optionally detected by contact sensors 167 and 177. The plurality of electrodes may enable a user to measure cardiac electric potentials with any of the above parts of their body regardless of the orientation of device 100 because the device may select the most appropriate pair or pairs of electrodes based on indicia of contact between the device and the user. This selection and measurement process is described in more detail below.

To simulate an electrocardiogram, it may be necessary to generate cardiac electrical activity signals from electric potentials between pairs of electrodes, or averages of those electric potentials, wherein a user contacts each pair with body parts from different sides of their body. By measuring the heart's electrical activity from both sides, device 100 may determine both the direction and magnitude of electrical impulses and in turn the characteristic waveforms necessary to simulate an electrocardiogram. For example, in FIG. 2, body parts 210 and 220 may contact a pair of electrodes used to form a cardiac electrical activity signal. If body part 210 were to be a user's right ring finger, for example, an efficacious choice of an upper body part 220 may be the user's left arm or one of the fingers on their left hand. In this manner, device 100 may measure the direction of the heart's electrical activity during the depolarization and/or repolarization phases of cardiac action potential from both the left side and the right side of the user's body, akin to a standard twelve-lead electrocardiogram which includes electrodes on both a patient's left and right arm.

Following the above example, a user may contact the device 100 with other body parts or combinations of body parts to obtain other heart electrical activity measurements that may be used to generate an electrocardiogram. For example, the lower body part 230 may be a user's right or left leg. In this manner, additional pairings of body parts 210 and 230 (for example, a user's right finger and a leg) and of body parts 220 and 230 (for example, a user's left arm or finger) may be formed, each of which may provide measurement of the depolarization and/or repolarization phases of cardiac action potential from above and below the user's heart in addition to measurement from the left and right side of the user's heart. In one or more examples, body parts 220 and 230 may include another portion of the user's body including their chest, hip, back, and/or shoulder. In this manner, it is apparent that a pair of electrodes used to generate a cardiac electrical activity signal may include, for example, an inner electrode 110 and an outer electrode 130, or two outer electrodes 130, for example contacting body parts 220 and 230 in FIG. 2.

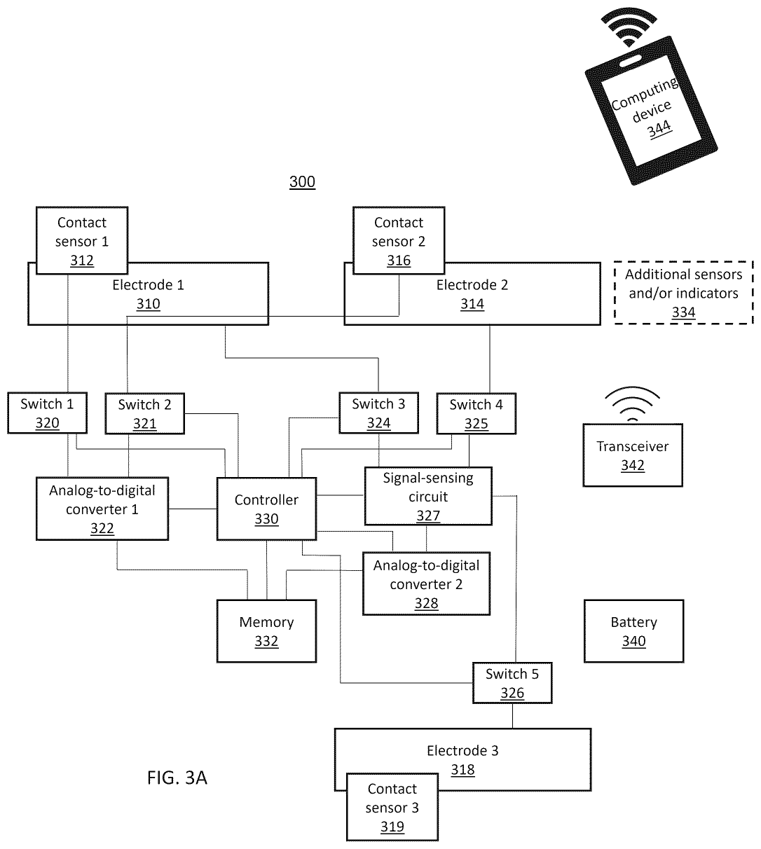

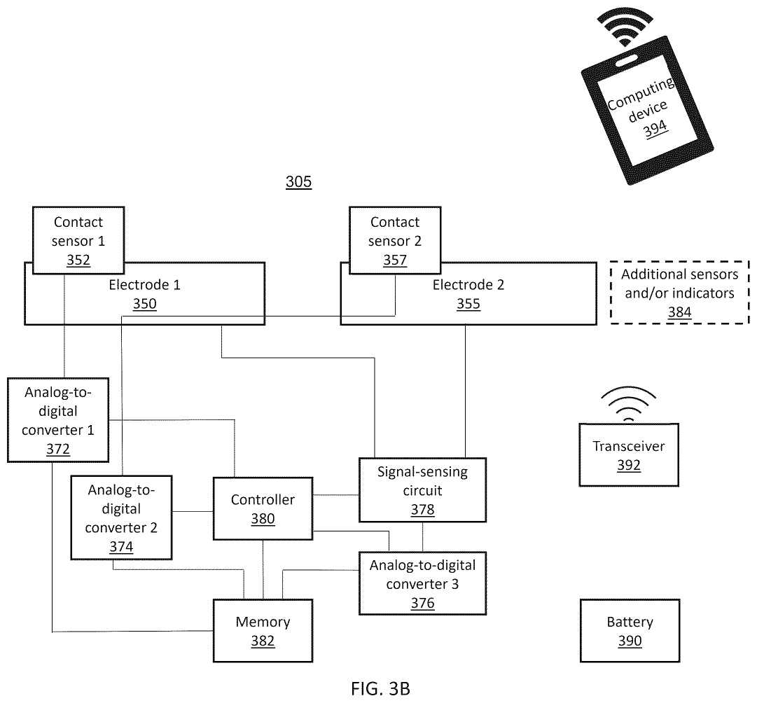

FIG. 3A depicts a circuit diagram of an exemplary wearable monitoring device 300, that may include one or more electrodes, for example, electrode 1 (310), electrode 2 (314), and electrode 3 (318). For example, electrode 1 (310) and electrode 2 (314) may be electrodes of an outer surface of the device and electrode 3 (318) may be an electrode of an inner surface of the device. Device 300 may include contact sensors 1, 2, and 3 (312, 316, and 319) that are each associated with a given electrode. Electrodes and contact sensors depicted may include inner electrodes 110 or outer electrodes 130 and contact sensors 115 shown in FIG. 1, respectively. Components depicted in FIG. 3A may be mounted to one or more printed circuit boards and located within housing 105. The device 300 may further include a controller 330 communicatively coupled to analog-to-digital converters 1 and 2 (322 and 328), signal-generating circuit 327, and/or electrical switches 1 to 6 (320, 321, 324, 325, and 326). Controller 330 may include one or more processors and memory storing instructions for execution by the one or more processors for causing the device 300 to perform the functionality described below. Electrical switches may include one or more transistors, communicatively coupled to electrodes 310 and 314 and/or to optional contact sensors 312 and 316 and may be used to control connection between the electrodes and/or the contact sensors and the analog-to-digital converters and/or signal-generating circuits.

In operation, device 300 may generate a cardiac electrical activity signal by measuring an electric potential between a selected pair of electrodes based on indicia of contact with a user. For example, device 300 (e.g., controller 330) may select electrodes 1 and 3 (310 and 318) for generating a cardiac electrical activity signal or may select electrodes 2 and 3 (310 and 318) for generating a cardiac electrical activity signal based on whether one or more indicia of contact with a user indicates that the user is contacting (or better contacting) electrode 1 or electrode 2.

In an example in which electrodes 1 and 3 (310 and 318) are selected for generating a cardiac electrical activity signal, controller 330 may close switches 3 and 5 (324 and 326) to ensure signal-generating circuit 327 may generate an analog signal corresponding to the electric potential difference between electrodes 1 and 3. This analog signal may then be converted to a digital signal by analog-to-digital converter 2 (328) before being sent to controller 330 and/or stored in memory 332. Device 300 may include additional electrodes which may be connected to signal-generating circuit 327 via one or more switches in an analogous manner to electrodes 1, 2, and 3. With multiple electrodes each connected via a switch to one or more signal-generating circuits, the controller 330 may, by controlling the state of each switch, control the connection between the signal-generating circuit and one or more pairs of electrodes to detect the electric potential between a selected pair of electrodes.

Optionally, controller 330 cycles through connections between pairs of electrodes and the signal-generating circuit(s) and may monitor the resulting electric potential signals measured between different pairs of electrodes for an indicia of contact between a user an a given pair of electrodes. For example, the controller 330 may process a given electric potential signal to detect a cardiac electrical activity signal (e.g., as opposed to noise). The detection of an indicia of contact can be the basis for selection of the particular pair of electrodes or electric potential signal as described in more detail below. If the controller does not detect cardiac electrical activity in a electric potential signal for a given pair of electrodes, the device may use the one or more switches to connect the signal-generating circuit to a different pair of electrodes.

Which electrode pairings to monitor and the order in which they are monitored may be based on signals from additional sensors, such as contact sensors. For example, if contact is detected via a contact sensor at an outer electrode, the device may choose a pair of electrodes for monitoring that include the outer electrode and the inner electrode opposite to the outer electrode as it is likely that user-applied pressure at the outer electrode would create contact at said inner electrode.

According to one or more embodiments, the device may measure electric potential difference between two electrodes by first measuring the electric potential difference between each electrode and a reference potential within the circuit of the device. The device may then subtract the electric potential difference between one electrode and the reference potential from the other to compute the electric potential difference between the two electrodes. For example, instead of directly measuring the electric potential between electrode 1 and electrode 3 in FIG. 3A, the device may connect electrode 1 and a reference point in the circuit to a signal-generating circuit to measure the electric potential between electrode 1 and the reference point, before connecting electrode 3 and the same reference point to the signal-generating circuit to measure the electric potential between electrode 3 and the reference, before subtracting one potential from the other to compute the electric potential between electrode 1 and electrode 3.

Contact sensors 312 and 316 may detect contact between the user and the device in the form of analog feedback signals. Controller 330 may cycle switches 1 and 2 (320 and 321) between their closed and open states to monitor the state of switches 1 and 2 to ensure analog signals corresponding to the level of contact between the user and each sensor may each in turn reach analog-to-digital converter 1 (322), before being sent to the controller 330 and/or stored in memory 332. For example, contact sensors 1 and 2 may be capacitive and/or inductive touch sensors, and the analog feedback signals may correspond to capacitance and/or inductance levels indicating the degree of contact or proximity of the user to each sensor, which may enable device 300 to choose the electrode associated with the contact sensor indicating the higher degree of contact. Thus, in the configuration shown in FIG. 3A, a single analog-to-digital converter may be used to monitor two contact sensors. Further, this concept may extend to configurations in which three or more contact sensors are connected to a single analog-to-digital converter.

In some implementations, switch 1 and 2 (320 and 321) may be combined into a single switch that controller 330 may use to cycle connection between analog-to-digital converter 1 (322) and each of switch 1 (320) and switch 2 (321). This single switch may include a plurality of inputs, connected in total to a plurality of contact sensors, enabling controller 330 to cycle connection between each contact sensor thereby monitoring the state of each sensor. In some implementations, contact sensors with built-in analog-to-digital converters may be used to output a digital signal thereby precluding the need for separate analog-to-digital converters as depicted in FIG. 3A.

FIG. 3B depicts a circuit diagram of an exemplary wearable monitoring device 305 configured to include a dedicated analog-to-digital converter for each of electrode 1 (350) and electrode 2 (355), thereby ensuring device 305 may continuously monitor the contact switches for evidence of contact by the user, in the form of a change in the analog signal. As shown, analog-to-digital converter 1 (372) may be dedicated to converting analog signals from contact sensor 1 (352), and analog-to-digital converter 2 (374) may be dedicated to contact sensor 2 (357). Likewise, signal-generating circuit 378 may be dedicated to monitoring the electric potential between electrode 1 (350) and electrode 2 (355), communicating the corresponding analog signal to analog-to-digital converter 3 (376). This configuration may be extended to additional contact sensors and electrodes, such that every contact sensor may have a dedicated analog-to-digital converter, optionally incorporated into the contact sensor itself, and such that one or more pairings of electrodes, optionally including all possible pairings of electrodes, may have a dedicated signal-generating circuit and analog-to-digital converter.

Such a configuration in which one or more pairings of electrodes, optionally including all possible pairings of electrodes, may have a dedicated signal-generating circuit and analog-to-digital converter may be used in implementations in which signals including electric potentials between one or more electrodes and/or one or more contact sensor feedback signals are monitored in real-time (i.e., operating on signals and/or data as they are generated), for example selecting one or more pairs of electrodes based on indicia of contact simultaneously. This configuration may also be used in implementations in which signals including electric potentials between one or more electrodes and/or one or more contact sensor feedback signals are recorded before generating one or more cardiac electrical activity signal as discussed below. While this recording-based configuration may reduce control complexity, it may also increase the required volume of the housing of the device as well as manufacturing costs. Thus, by using switches to connect a single analog-to-digital converter and/or signal-generating circuit to multiple contact sensors and/or electrodes, a manufacturer may reduce the required device housing volume and/or part and assembly costs.

Regardless of the presence or number of switches, analog-to-digital converters may be communicatively coupled to a controller, shown as 330 in FIGS. 3A and 380 in FIG. 3B and/or a memory 332 or 382 of the wearable device to enable control and storage of, for example, electric potential data detected by the one or more electrode pairs following conversion of the detected analog signals to digital signals. The wearable device may directly record in the memory one or more electric potentials between electrodes (optionally including electric potentials corresponding to all possible electrode pairings) after conversion to digital signals and before selection of electric potential signals based on indicia of contact as described below. Alternatively or additionally, the wearable device may instead select one or more electrode pairs first based on one or more indicia of contact before generating cardiac electrical activity signals and recording in the memory as described below. The wearable device may additionally include battery 340 or 390 to power the above-mentioned electrical components wherein the battery may be removeable and replaceable, and/or rechargeable, allowing a user to monitor their cardiac electrical activity using the device without connection to an external source of power.

The wearable device may additionally include a transceiver 342 or 392 that may transmit one or more data types to a computing device 344 or 394 communicatively coupled to the device. The computing device may take the form of the user's mobile phone, tablet, and/or personal computer, for example. Transmitted data types may include, among others, selected pairs of electrodes and associated electric potential data, generated cardiac electrical activity signals, and/or data corresponding to an electrocardiogram simulation, and may be transmitted to an application associated with the device or to a storage space within the computing device. According to one or more examples, the wearable device may transmit via the transceiver data that includes one or more generated cardiac electrical activity signals to the communicatively coupled computing device, enabling the coupled device to the use one or more techniques, discussed below, to simulate an electrocardiogram. According to one or more embodiments, the wearable device may transmit, via the transceiver, data corresponding to a partially or entirely complete electrocardiogram simulation to the communicatively coupled computing device, enabling the coupled device to complete the simulation and/or display the results for the user's review.

The computing coupled may optionally be used for input by the user, for example by selecting one or more locations, on the user's body and on the device, at which the user is contacting the device via drop down menu or by tapping on a selectable diagram of the human body and/or electrode locations on the device. Such selections may optionally be prioritized over indicia of contact detections described below allowing user input to guide selection of pairs of electrodes. Additionally, the above-described prompts for the user, for example resulting from poor contact with an electrode or from contacting the wearable device at two or more points not on opposite sides of the heart, may be provided as visual and/or audible prompts via the coupled device.

According to one or more embodiments, the wearable device may further include additional sensors and/or indicators 334 or 384 for example an accelerometer to measure user motion, an infrared sensor to measure user body temperature, a pulse oximetry sensor to measure user heart rate and/or blood oxygen saturation, and/or an LED light to communicate the status of the device for example relating to battery power, connection to external computing devices, and/or contact with the user at one or more electrodes. Referring again to FIG. 1, one or more of these additional sensors and/or indicators may be incorporated within housing 105 of device 100, at inward-facing surface 125 proximate to one or more inner electrodes 110, and/or at the outward-facing surface proximate to one or more outer electrodes 130, with location of each sensor or indicator optionally varying based on the type and/or function of a particular additional sensor or indicator.

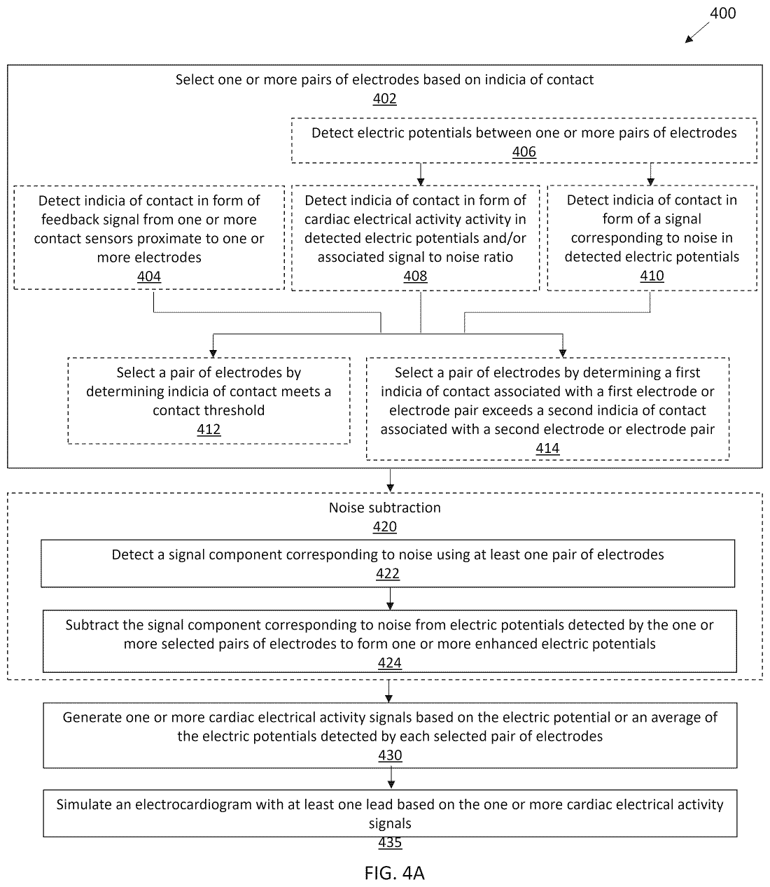

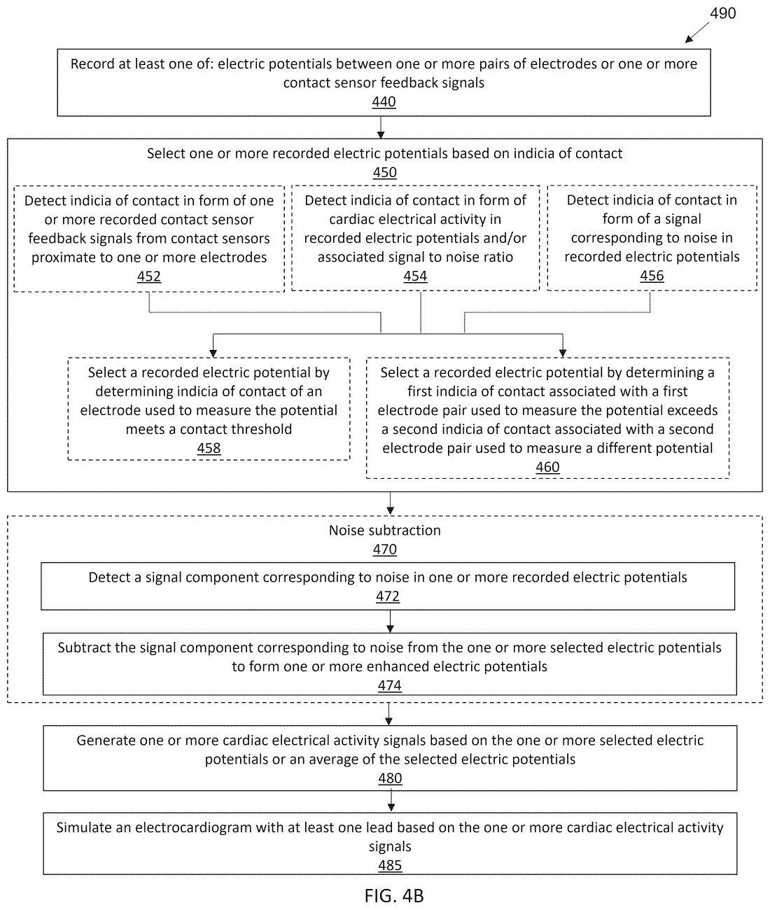

FIG. 4A depicts an exemplary method 400 for generating data related to electrocardiography using a wearable device. The device may detect the electric potential between one or more pairs of electrodes in the form of an analog signal generated by a signal-generating circuit. Electric potentials between pairs of electrodes may be used to interpret cardiac electrical activity such as the depolarization phase of the cardiac action potential as discussed above. These analog electric potentials signals may be converted to digital signals using one or more analog-to-digital converters as discussed above. Some or all steps of method 400 may be performed, for example, by wearable device 100, or wearable device 300 (e.g., by controller 330), alone, or in conjunction with an external computing device communicatively connected to the wearable device, such as computing device 344 and/or computing device 394.

At step 402, the wearable device (e.g., controller 330) may select one or more pairs of electrodes to use for cardiac electrical activity monitoring based on one or more indications of contact between a user and the one or more electrodes. By selecting pairs of electrodes before proceeding to generate one or more cardiac electrical activity signals, method 400 may reduce enable simpler circuitry, reduce computational resource consumption, conserve battery power, and store only the data necessary to produce the data that may be used to simulate an electrocardiogram.

Selection of one or more pairs of electrodes at step 402 may include two sub-steps—detection of one or more indications of contact and selection of one or more pairs of electrodes on the basis of the indicia of contact. One or more of these sub-steps may include one or more optional detection or selection techniques.

One or more indications of contact between a user and an electrode or pair of electrodes, referred to as indicia of contact, may be detected by the device. For example, at step 404, the device may use a feedback signal from one or more contact sensors 115 that may be co-located and associated with one or more inner electrodes 110 and/or outer electrodes 130. As shown in FIGS. 1 and 2, co-location of contact sensors 115 with one or more electrodes 110 and/or 130 may enable the device to detect contact between the user and one or more of the electrodes. As mentioned, contact sensors 115 may include capacitive and/or inductive touch sensors, limit switches, and/or optical and/or ultrasonic-based proximity sensors. At step 404, with capacitive touch sensors serving as an exemplary embodiment of the one or more contact sensors 115, the device may detect indicia of contact in the form of a feedback signal from one or more capacitive touch sensors indicating a user's contact with one or more associated electrodes.

Once an indicia of contact is detected at one electrode, the device may automatically check for indicia of contact on the opposite surface of the device given symmetries inherent in the design of the device. For example, as shown in FIG. 2, if indicia of contact is detected at the uppermost outer electrode 165 corresponding to pressure from body part 220, the device may infer that the pressure creating the contact is creating contact between the user and the uppermost inner electrode 155 and automatically check for indica of contact at this electrode. In the example of FIG. 2, this pressure indeed is creating contact between the uppermost inner electrode 155 and body part 210.

Alternatively or additionally, the device may at step 406 first detect the electric potential between one or more pairs of electrodes, then detect one or more indicia of contact based on the detected electric potentials. As mentioned in the context of FIGS. 3A and 3B, the device may change the state of switches connected to one or more electrodes to vary the pair of electrodes a particular signal-generating circuit is connected to before measuring the potential between the pair of electrodes and converting the signal from analog to digital form using an analog-to-digital converter. By cycling through multiple pairs of electrodes, optionally based on external inputs such as feedback signals from contact sensors, the device may check for indicia of contact within the detected electric potentials between electrodes forming multiple pairs of electrodes.

At step 408, the device may use these detected electric potentials to detect indicia of contact in the form of cardiac electrical activity, such as activity corresponding to the cardiac action potential. That is, the device may infer that a user has made contact with a pair of electrodes because the electric potential signal measured between the pair of electrodes reflects electrical activity associated with the user's heart. Indicia of contact determined in this manner may be contingent on the detected signal to noise ratio in the electric potential data being sufficiently low, i.e., low enough that the cardiac electrical activity component of the electric potential data may be usable for downstream processes such as the simulation of an electrocardiogram.

At step 410, indicia of contact may alternatively or additionally include detection by the device of noise in the detected electric potential signals measured between one or more pairs of electrodes, allowing the inference that a user is not making contact with one or both electrodes of the pair or making contact that is insufficient for cardiac electrical activity monitoring. That is, by not detecting cardiac electrical activity, and by detecting only noise at one or more pairs of electrodes, the device may infer that a user is not sufficiently contacting one or both electrodes, and may thus not consider one or both of those electrodes when selecting one or more pairs of electrodes, during time periods corresponding to the noise measurement.

With one or more of the methods of steps 404, 408, and 410 used to detect indicia of contact corresponding to one or more electrodes or pairs of electrodes, the device may next select one or more pairs of electrodes for further processing based on the detected indicia of contact. In the event the device detects a user is contacting two electrodes equally with the same part of their body, the device may optionally average the detected electric potentials detected at each electrode or may choose one signal based on one or more the indicia of contact approaches described above.

At step 412, this selection may optionally take the form of a determination that detected indicia of contact meet a contact threshold. For example, in the case in which feedback signals from capacitive and/or inductive touch sensors form the indicia of contact, the device may require that the detected shift in capacitance and/or inductance exceed the contract threshold, i.e. require that the user is sufficiently proximate or applying sufficient pressure to the capacitive and/or inductive touch sensor and/or electrode, before the indicia of contact is determined to indicate contact sufficient to select an associated electrode to form an electrode pair. In the case in which detected cardiac electrical activity optionally including an associated signal to noise ratio forms the indicia of contact, for example, the device may require the signal to noise ratio to exceed a contact threshold before the indicia of contact is determined to indicate contact sufficient to select an associated electrode pair. Finally, in the example in which detection of a signal corresponding to noise forms the indicia of contact, the device may require that the noise amplitude of the signal exceed a contact threshold before the indicia of contact is determined to indicate an absence of contact sufficient to exclude from selection an associated electrode or electrode pair.

At step 414, the device may additionally or alternatively compare indicia of contact associated with one electrode and/or electrode pair with the indicia of contact associated with one or more other electrodes and/or electrode pairs, and select the electrode and/or electrode pair with an indicia of contact higher than that of one or more other electrodes and/or electrode pairs. For example, if capacitance feedback from a first capacitive touch sensor associated with a first inner electrode 110 exceeds the capacitance value from a second capacitive touch sensor associated with a second inner electrode, the device may select the first inner electrode to form a pair of electrodes given that its higher indica of contact may improve the quality of any detected electric cardiac electrical activity. Further, if the signal to noise ratio associated with an electric potential signal corresponding to cardiac electrical activity detected by a first pair of electrodes exceeds that of an electric potential signal corresponding to cardiac electrical activity detected by a second pair of electrodes, the device may select the first pair to improve signal quality.

As mentioned, the comparative technique of step 414 may be used not only as an alternative to but optionally in addition to the threshold technique of step 412. For example, with indicia of contact from capacitive touch sensors associated with two or more electrodes available, the device may require not only that the selected electrode be associated with an indica of contact that is higher than that associated with one or more other electrodes but that the indica also exceed a contact threshold.

At step 420, the device may subtract from one or more cardiac electrical activity signals common-mode noise, or components of noise in electric potential data detected by one or more pairs composed of inner electrodes 110 and/or outer electrodes 130, thereby improving signal quality and the fidelity of any simulated electrocardiograms based on the cardiac electrical activity signals. At step 422, the device may detect a signal component corresponding to noise in one or more electric potential signals. If a signal component corresponding to noise is detected at two or more electrode pairs, the device may compute the average of the signal components to form a single signal component to be used for further processing. At step 424, this signal component or components may be subtracted from detected electric potentials associated with each pair of electrodes selected at step 402, thereby forming noise-reduced, enhanced electric potentials. To assist with signal component detection and subtraction, the device may optionally use one or more machine learning techniques such as denoising algorithms trained on electrocardiography datasets including significant noise and/or artifact and interference detection and removal algorithms.

Once indicia of contact have been detected, used to select one or more pairs of electrodes, and optionally following noise reduction, at step 430, the device may use the one or more detected electric potentials or enhanced electric potentials to generate one or more cardiac electrical activity signals. To generate one or more cardiac electrical activity signals, the device may use electric potential data or enhanced electric potential data as detected or may amplify one or more electric potentials to match other electric potential values or to meet a minimum amplitude value. In examples in which a user contacts three or more electrodes, generation of a cardiac electrical activity signal may also include generating an augmented cardiac electrical activity signal by computing the average of two electric potentials. For example, a first electric potential representing the potential difference between a user's left arm and right arm may be averaged with a second electric potential representing the potential difference between a user's left leg and right arm to form the basis of an augmented cardiac electrical activity signal. In this manner, each cardiac electrical activity signal may form data associated with one or more “leads” or augmented leads of a traditional electrocardiogram.

In generating one or more cardiac electrical activity signals, the device may utilize signal processing techniques and/or machine learning algorithms to improve signal quality and ensure representation of relevant waveforms and other signal features corresponding to cardiac electrical activity. Signal processing techniques involved may include electrocardiogram waveform analysis and/or time-frequency analysis such as Fourier transforms. Machine learning techniques may be trained on large electrocardiography datasets collected from patients with normal and/or abnormal physiology using lead locations varied as a function of position relative to the patient's heart and/or standard lead locations associated with standard full (12-lead) electrocardiograms, which may allow device 100 to infer the surface anatomic location at which the user is contacting the device through nonlinear interpolative techniques.

Such datasets may be preprocessed by applying principal component analysis to reduce dimensionality and/or used to train neural network-based machine learning models. Such neural networks may associate cardiac electrical activity signal patterns in the datasets with corresponding lead locations, thereby assisting device 100 in detecting cardiac electrical activity and/or predicting locations at which the user is contacting the device. In addition to or as an alternative to neural networks, models based on support vector classifiers may be trained based on such electrocardiography datasets to recognize patterns associated with various lead locations thereby assisting device 100 in predicting locations at which the user is contacting the device.

To improve its ability to recognize cardiac electrical activity and/or predict locations at which the user is contacting the device, device 100 may reference electrocardiography data captured during a 12-lead electrocardiogram provided by the user. Such an electrocardiogram may correspond to a user's nominal or baseline cardiac state, and/or may correspond to their cardiac state during certain clinical conditions, for example periods during which a user's pacemaker is active and/or a cardiovascular symptom is present. Such data may enable customized training of device 100, thereby improving its ability to detect cardiac signals and/or localize the one or more points of the user's body contacting one or more electrodes of the device.

In the event that a cardiac electrical activity signal cannot be generated from the electric potential measured between a pair of electrodes, the device or an associated computing device such as an application on a mobile phone or computer communicatively coupled to the device may provide recommendations for corrective actions the user may take. For example, if a previously detected cardiac electrical activity signal is no longer present, and optionally if one or more of the above-described indicia of contact indicate contact has been reduced, the device may recommend the user check contact with one or more electrodes of the device or increase pressure to recreate contact. If a noise signal or an uninterpretable cardiac electrical activity signal is detected, and optionally if indicia of contact indicate contact is present, the device may infer that the two parts of the user's body contacting the pair of electrodes are on the same side of the heart, a configuration that may preclude computation of the direction and magnitude of electrical impulses from the heart as discussed above. In this case, the device may prompt the user to switch one of the parts of their body contacting the device to the opposite side. For example, if the device is being worn on a user's right finger, with one or more inner electrodes 110 contacting their finger, and the user is touching one or more outer electrodes 130 to the user's right shoulder, the device may prompt the user to instead contact outer electrodes 130 with their left leg instead, thereby allowing measurement of electrical cardiac electrical activity from opposite sides of the heart.

Once the device generates one or more cardiac electrical activity signals, at step 435, the device may optionally use said cardiac electrical activity signals to simulate an electrocardiogram. As discussed, cardiac electrical activity signals generated based on electric potential detected between a pair of electrodes ideally on opposite sides of the heart may simulate a lead of a traditional electrocardiogram, including waveforms such as the P wave, QRS complex, and T wave that represent different phases of the cardiac action potential. As discussed in the context of FIG. 2, multiple cardiac electrical activity signals may be used to provide further perspective on the direction and magnitude of the heart's electrical activity, for example, three or more cardiac electrical activity signals may be used. As discussed, three portions of the user's body used to form three pairs of electrodes may include, for example, a user's right finger, leg, and left arm or finger, and may correspond to body parts 210, 220, and 230 shown in FIG. 2. Additional cardiac electrical activity signals may be formed by taking advantage of the plurality of outer electrodes 130 and/or inner electrodes 110. According to one or more embodiments, an external computing device communicatively coupled to the device may alternatively or additionally simulate an electrocardiogram using the generated one or more cardiac electrical activity signals, as discussed in greater depth below.

FIG. 4B depicts an exemplary method 490 for generating data related to electrocardiography using a wearable device. Method 490 may be performed in addition to or as an alternative to method 400 of FIG. 4A. Some or all steps of method 490 may be performed, for example, by wearable device 100 or wearable device 305 (e.g., by controller 380), alone, or in conjunction with an external computing device communicatively connected to the wearable device, such as computing device 344 and/or computing device 394. Method 490 includes recording multiple electrical potential signals corresponding to multiple different electrode pairs and selecting, from among the multiple electrical potential signals, one or more cardiac electrical activity signals.

At step 440, signals corresponding to electric potentials between multiple different pairs of electrodes may be recorded in the memory of the device. For example, with a wearable device having one inner electrode and four outer electrodes, an electric potential can be recorded for each combination of the inner electrode with one of the outer electrodes (for a total of four electric potential signals). Optionally, electric potential signals from electrode pairs formed from all combinations of electrodes may be recorded in the memory of the device.

Step 440 may include recording signals from one or more contact sensors in the memory of the device. For example, signals from a plurality of different contact sensors (e.g., some or all of the contact sensors of the device) may be recorded in the memory synchronously with the electric potential signals. At this step, selection of which pair(s) of electrodes to use has not been made. As such, not every one of the recorded signals captures (or captures well) cardiac electrical activity monitoring. For example, a given recorded signal may correspond to an electrode that does not make any contact with a user.

At step 450, one or more of the electric potential signals stored in the memory of the device are selected based on indicia of contact between electrodes of the device and a user. Thus, step 450 includes discriminating from among the recorded electric potential signals from step 440 to determine which capture (or better capture) cardiac electric activity. Step 450 may be performed periodically, such as after a predefined period of time (which may be programmable) has elapsed. For example, step 450 may be performed every 30 minutes. Step 450 may be performed periodically based on a proportion of the storage capacity of the memory of the device. For example, step 450 may be performed once the storage capacity of the device (or the proportion of the storage capacity dedicated to recording electric potentials) is at 80 percent. Non-selected electric potentials recorded in the memory may then be deleted to make room for new recordings.

Method 490 may be asynchronous, operating on a pre-recorded set of data (step 440), which may contrast with method 400 of FIG. 4A, which may occur in real-time (i.e., operating on signals and/or data as they are generated). As explained above, step 450 generally includes generating a recorded dataset that includes multiple electric potential signals generated using a plurality of different sets of electrodes (and which may include recorded contact sensor feedback signals) and then selecting which of those recorded signals to use for generating one or more cardiac electrical activity signals (which may be used for generating an electrocardiogram) based on indicia of contact between a user and electrodes. Aspects of method 490 involving detection in the recorded data of indicia of contact via one or more contact sensor feedback signals (e.g., step 452), detection of cardiac electrical activity or the signal to noise ratio thereof (e.g., step 454), and detection of an absence of contact via a signal corresponding to noise (e.g., step 456) may be similar to steps 404, 408, and 410 of FIG. 4A, and thus, their details are not repeated here for brevity. Further, various steps in FIG. 4B involving selection of recorded electric potentials via determination that the indicia of contact of an associated electrode or electrode pair meets a contact threshold (e.g., step 458) and determination that an indicia of contact of a first electrode pair used to measure a potential signal exceeds an indicia of contact of a second electrode pair used to measure a potential signal (e.g., step 460) may be similar to analogous steps of method 400, such as steps 412 and 414, and their details are not repeated here for brevity. Additionally, steps corresponding to noise subtraction (e.g., step 470), including detecting a signal component corresponding to noise in the recorded electric potentials at step 472 and subtracting the signal component corresponding to noise from the one or more selected electric potentials at step 474, as well as generation one or more cardiac electrical activity signals based on the one or more selected electric potentials at step 480, and simulation of an electrocardiogram at step 485 may be similar to steps 420, 422, 424, 430, and 435 of FIG. 4A respectively, and as such, their details are not repeated here for brevity.

According to one or more embodiments, the device may use a combination of the above two approaches. For example, the device may record electric potentials between one or more pairs of electrodes, optionally formed using all electrodes of the device, and optionally including one or more contact sensor feedback signals. The device may record while also selecting one or more pairs of electrodes, optionally subtracting noise, and generating one or more cardiac electrical activity signals. These cardiac electrical activity signals may then be used to display, on an external computing device communicatively coupled to the device, a real-time electrocardiogram. The device and external computing device may then finalize the data, and in so doing may refer to the recorded electric potential and/or contact sensor feedback signals in case one or more electric potentials not measured in real-time produced a higher fidelity signal or a signal corresponding to a lower signal to noise ratio.

Thus, in the above-described manner, the device may monitor electrical activity of the heart in a continuous manner, detecting electric potentials between one or more pairs of electrodes contacting the user, confirming contact is made and/or cardiac electrical activity is detected by forming one or more indicia of contact and selecting one or more pairs of electrodes on this basis, optionally reducing common-mode noise detected before generating one or more cardiac electrical activity signals based on detected electric potentials. These one or more cardiac electrical activity signals may then serve as the basis of a simulated electrocardiogram involving one or more leads that may be computed in part or whole on the device or transmitted to a communicatively coupled computing device to make or complete the computation and display the result for user review. According to one or more embodiments, as discussed above, the device may also batch and store electric potentials detected between one or more pairs of electrodes contacting the user, thus allowing the remainder of the method steps, including analysis of contact and generation of one or more cardiac electrical activity signals, to occur asynchronously, following collection of data for a user-defined time period and/or until a proportion of the storage capacity associated with the memory of the device is reached.

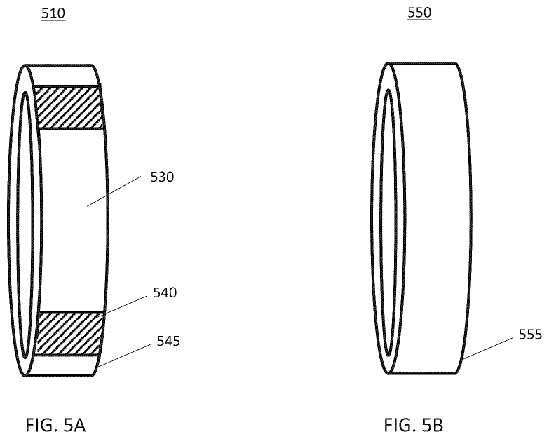

FIG. 5A depicts a three-dimensional view of a configuration 510 of a wearable monitoring device wherein outer electrodes 530 and outer electrical insulators 540 may be visually demarcated, enabling a user to discern the locations of outer electrodes 530 and thus use as a reference when contacting outward-facing surface 545 of the device with one or more parts of their body, thereby potentially improving electrode-body contact and the quality of detected electric potentials.

FIG. 5B depicts a three-dimensional view of an additional or alternative configuration 550 of wearable monitoring the device wherein outer electrodes and outer electrical insulators may not be visually demarcated, giving outward-facing surface 555 of the device a uniform appearance. While not providing the user an indication of where to make contact with the device, embodiment 550 does provide a potentially more visually appealing exterior and may allow for application of opaque protective layers or coatings.

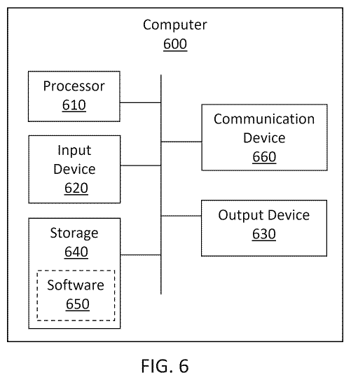

In one or more examples, one or more of the disclosed systems and methods utilize or may include a computing system. FIG. 6 depicts an exemplary computing system 600 according to one or more examples of the disclosure that may be used for one or more systems disclosed herein, such as computing device 344 or computing device 394, and/or utilized for performing one or more methods described herein, such as method 400 of FIG. 4A and/or method 490 of FIG. 4B. Computing system 600 can be a host computer connected to a network. Computing system 600 can be a client computer or a server. As shown in FIG. 6, computing system 600 can be any suitable type of microprocessor-based device, such as a personal computer, workstation, server, or handheld computing device, such as a phone or tablet. The computer can include, for example, one or more of processor 610, input device 620, output device 630, storage 640, and communication device 660. Input device 620 and output device 630 can correspond to those described above and can either be connectable or integrated with the computer.

Input device 620 can be any suitable device that provides input, such as a touch screen or monitor, keyboard, mouse, or voice-recognition device. Output device 630 can be any suitable device that provides an output, such as a touch screen, monitor, printer, disk drive, or speaker.

Storage 640 can be any suitable device that provides storage, such as an electrical, magnetic, or optical memory, including a random-access memory (RAM), cache, hard drive, CD-ROM drive, tape drive, or removable storage disk. Communication device 660 can include any suitable device capable of transmitting and receiving signals over a network, such as a network interface chip or card. The components of the computer can be connected in any suitable manner, such as via a physical bus or wirelessly. Storage 640 can be a non-transitory computer-readable storage medium comprising one or more programs, which, when executed by one or more processors, such as processor 610, cause the one or more processors to execute methods described herein.

Software 650, which can be stored in storage 640 and executed by processor 610, can include, for example, the programming that embodies the functionality of the present disclosure (e.g., as embodied in the systems, computers, servers, and/or devices as described above). In one or more examples, software 650 can include a combination of servers such as application servers and database servers.

Software 650 can also be stored and/or transported within any computer-readable storage medium for use by or in connection with an instruction execution system, apparatus, or device, such as those detailed above, that can fetch and execute instructions associated with the software from the instruction execution system, apparatus, or device. In the context of this disclosure, a computer-readable storage medium can be any medium, such as storage 640, that can contain or store programming for use by or in connection with an instruction execution system, apparatus, or device.

Software 650 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include but is not limited to, an electronic, magnetic, optical, electromagnetic, or infrared wired or wireless propagation medium.

Computing system 600 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computing system 600 can implement any operating system suitable for operating on the network. Software 650 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The foregoing description, for the purpose of explanation, has been described with reference to specific embodiments and/or examples. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A wearable electronic device for cardiac electrical activity monitoring, the device comprising:
a housing configured to be worn around a first body part of a user, the housing having an inward-facing surface that faces the first body part and an outward-facing surface that faces away from the first body part;
a plurality of electrodes comprising:
at least one inner electrode at the inward-facing surface of the housing for contacting the first body part;
multiple outer electrodes at the outward-facing surface of the housing for contacting one or more other parts of a body of the user; and
a controller configured to generate a cardiac electrical activity signal using a pair of electrodes selected from the plurality of electrodes based on at least one indicia of contact between an electrode of the pair of electrodes and the user.

2. The device of claim 1, wherein the controller is configured to generate multiple cardiac electrical activity signals using multiple pairs of the electrodes and the multiple cardiac electrical activity signals comprise:
a first cardiac electrical activity signal that is based on an electric potential between a first pair of electrodes, a second cardiac electrical activity signal that is based on an electric potential between a second pair of electrodes, and a third cardiac electrical activity signal that is based on the first cardiac electrical activity signal and the second cardiac electrical activity signal,
wherein the first pair of electrodes comprises an inner electrode that contacts the first body part and a first outer electrode that contacts a second body part, and wherein the second pair of electrodes comprises the inner electrode and a second outer electrode that contacts a third body part.

3. The device of claim 1, wherein:
the controller is configured to select the pair of electrodes from the plurality of electrodes by determining that a first indicia of contact associated with a first electrode of the plurality of electrodes exceeds a second indicia of contact associated with a second electrode of the plurality of electrodes, and
the first electrode and the second electrode are both located at the inward-facing surface or both located at the outward-facing surface of the housing.

4. The device of claim 1, further comprising at least one contact sensor configured to detect contact with the user, wherein the at least one indicia of contact comprises a feedback signal from the at least one contact sensor.

5. The device of claim 4, wherein the at least one contact sensor comprises multiple contact sensors, and wherein each contact sensor of the multiple contact sensors is proximate to a respective electrode of the plurality of electrodes.

6. The device of claim 5, further comprising at least one analog-to-digital converter and at least one switch for selectively connecting different contact sensors to the at least one analog-to-digital converter.

7. The device of claim 4, wherein the at least one contact sensor comprises at least one capacitive touch sensor.

8. The device of claim 1, comprising at least one signal-generating circuit and at least one switch for selectively connecting different electrodes to the at least one signal-generating circuit.

9. The device of claim 1, wherein the pair of electrodes comprises an inner electrode opposite to an outer electrode, wherein the inner electrode and/or the outer electrode was selected based on the at least one indicia of contact.

10. The device of claim 1, wherein the outward-facing surface of the ring comprises an indication of a position of at least one of the multiple outer electrodes.

11. The device of claim 1, wherein the outward-facing surface of the ring does not comprise an indication of positions of any of the multiple outer electrodes.

12. The device of claim 1, wherein the device is configured as a ring and the first body part is a finger.

13. A method for cardiac electrical activity monitoring using a wearable electronic device, the method comprising:
selecting a pair of electrodes from a plurality of electrodes of the device based on at least one indicia of contact between an electrode of the pair of electrodes and a user; and
generating a cardiac electrical activity signal using the pair of electrodes.

14. The method of claim 13, further comprising generating multiple cardiac electrical activity signals using multiple pairs of electrodes.

15. The method of claim 14, wherein generating multiple cardiac electrical activity signals comprises:
generating a first cardiac electrical activity signal based on an electric potential between a first pair of electrodes;
generating a second cardiac electrical activity signal based on an electric potential between a second pair of electrodes; and
generating a third cardiac electrical activity signal based on the first cardiac electrical activity signal and the second cardiac electrical activity signal,
wherein the first pair of electrodes comprises an inner electrode that contacts the first body part and a first outer electrode that contacts a second body part, and wherein the second pair of electrodes comprises the inner electrode and a second outer electrode that contacts a third body part.

16. The method of claim 15, further comprising generating data to simulate an electrocardiogram with at least three leads based on at least one of: the first cardiac electrical activity signal, the second cardiac electrical activity signal, or the third cardiac electrical activity signal.

17. The method of claim 13, wherein selecting the pair of electrodes from the plurality of electrodes comprises determining that the at least one indicia of contact meets a threshold value.

18. The method of claim 13, wherein selecting the pair of electrodes from the plurality of electrodes comprises determining that a first indicia of contact associated with a first electrode of the plurality of electrodes exceeds a second indicia of contact associated with a second electrode of the plurality of electrodes.

19. The method of claim 13, wherein selecting the pair of electrodes from the plurality of electrodes comprises detecting contact with the user using at least one contact sensor, wherein the at least one indicia of contact comprises a feedback signal from the at least one contact sensor.

20. The method of claim 13, wherein selecting the pair of electrodes from the plurality of electrodes comprises detecting cardiac electrical activity using the pair of electrodes and/or detecting a signal to noise ratio corresponding to the detection of cardiac electrical activity using the pair of electrodes.

21. The method of claim 13, wherein the pair of electrodes is a first pair of electrodes and wherein selecting the pair of electrodes from the plurality of electrodes comprises detecting a signal corresponding to noise using a second pair of electrodes.

22. The method of claim 13, wherein the pair of electrodes is a first pair of electrodes, and wherein generating the cardiac electrical activity signal comprises enhancing a signal generated by the first pair of electrodes based on a signal generated by a second pair of electrodes of the plurality of electrodes.

23. The method of claim 22, wherein the signal generated by the second pair of electrodes corresponds to noise, and wherein enhancing the signal generated by the first pair of electrodes comprises subtracting the signal generated by the second pair of electrodes.

24. The method of claim 13, further comprising generating data to simulate an electrocardiogram with at least one lead based on the cardiac electrical activity signal.

25. A method for cardiac electrical activity monitoring using a wearable electronic device, the method comprising:
recording multiple signals using multiple pairs of electrodes of the device; and
selecting at least one signal as a cardiac electrical activity signal from the multiple signals based on at least one indicia of contact between a user and an electrode of a pair of electrodes used to measure the signal.

26. The method of claim 25, wherein selecting the at least one signal from the multiple signals comprises determining that the at least one indicia of contact meets a threshold value.

27. The method of claim 25, wherein the at least one signal comprises a first signal, and wherein selecting the first signal from the multiple signals comprises determining that a first indicia of contact associated with the pair of electrodes used to measure the first signal exceeds a second indicia of contact associated with a pair of electrodes used to measure the second signal.

28. The method of claim 25, further comprising recording at least one contact sensor feedback signal from one or more contact sensors, wherein the at least one indicia of contact comprises the at least one recorded contact sensor feedback signal.

29. The method of claim 25, wherein selecting the at least one signal from the multiple signals comprises detecting cardiac electrical activity and/or detecting a signal to noise ratio corresponding to the detection of cardiac electrical activity.

30. The method of claim 25, wherein the pair of electrodes is a first pair of electrodes and wherein selecting the at least one signal from the multiple signals comprises detecting a signal corresponding to noise using a second pair of electrodes.

31. The method of claim 25, wherein the pair of electrodes is a first pair of electrodes and wherein the method further comprises enhancing a signal generated by the first pair of electrodes based on a signal generated by a second pair of electrodes of the plurality of electrodes.

32. The method of claim 31, wherein the signal generated by the second pair of electrodes corresponds to noise, and wherein enhancing the signal generated by the first pair of electrodes comprises subtracting the signal generated by the second pair of electrodes.

33. The method of claim 25, further comprising generating data to simulate an electrocardiogram with at least one lead based on the cardiac electrical activity signal.

34. The method of claim 25, further comprising:
generating a first cardiac electrical activity signal based on an electric potential between a first pair of electrodes;
generating a second cardiac electrical activity signal based on an electric potential between a second pair of electrodes; and
generating a third cardiac electrical activity signal based on the first cardiac electrical activity signal and the second cardiac electrical activity signal;
wherein the first pair of electrodes comprises an inner electrode that contacts the first body part and a first outer electrode that contacts a second body part, and wherein the second pair of electrodes comprises the inner electrode and a second outer electrode that contacts a third body part.

35. The method of claim 25, further comprising generating data to simulate an electrocardiogram with at least three leads based on at least one of: the first cardiac electrical activity signal, the second cardiac electrical activity signal, and the third cardiac electrical activity signal.
